(19) United States
(12) Patent Application Publication
(10) Pub. No.: US 2021/0158964 A1
(43) Pub. Date: May 27, 2021

(54) HEALTH TESTS BY PERSONAL HEALTH CARE SYSTEMS

(71) Applicant: Fresenius Medical Care Holdings, Inc., Waltham, MA (US)

(72) Inventors: David Yuds, Hudson, NH (US); Samiullah K. Durrani, Harvard, MA (US)

(21) Appl. No.: 16/692,360

(22) Filed: Nov. 22, 2019

(51) Int. Cl. G16H 50/30; G16H 10/60; G16H 15/00; A61B 5/22; A61B 5/00; A61B 5/12; A61B 5/145; A61B 5/20; A61B 3/08

(52) U.S. Cl. CPC G16H 50/30; G16H 10/60; G16H 15/00; A61B 5/225; A61B 3/08; A61B 5/0022; A61B 5/123; A61B 5/14525; A61B 5/20; A61B 5/6866

(57) ABSTRACT

A systems is provided herein to evaluate health of a patient. The system includes one or more testing modules configured to receive examination data related to at least one of gripping power, vision health, or hearing strength of a user; one or more pumps configured to provide fluid to or receive fluid from the user's body and analyze the fluid to determine one or more contents included in the fluid; a data processor unit configured to analyze the received examination data to determine whether the user is at risk of a health problem, and a communication unit capable of communicating information to a computing system. In response to determining that the user is at risk of the health problem, the data processor requests the communication unit to notify a health care provider about the risk. Corresponding methods and non-transitory computer readable medium are also disclosed.

### TECHNICAL FIELD

This invention relates to methods and medical devices that can be used to measure overall health of patients outside health care facilities, e.g., at a patient's home.

### BACKGROUND

Personal health care systems, such as home dialysis machines, have provided patients the convenience of monitoring and controlling their chronic diseases at home and have eliminated a need for frequent visits to medical clinics for such purposes. Most personal health care systems focus on particular chronic diseases without paying attention to other vital signs of the patients. Ignoring such signs can cause a patient to not be able to use a personal health care system properly.

### SUMMARY

Implementations of the present disclosure include computer-implemented methods and systems for evaluating health of a user by using a personal health care system that is capable of evaluating, monitoring and/or providing patients with assistance in chronic diseases such as diabetes, high blood pressure, physical disabilities, etc. The methods and systems described herein test different health factors, such as age, sex, gripping power, hearing strength, visual capabilities, etc. by a personal health care system such as a peritoneal dialysis system, a hemodialysis system, etc.

A typical health care system focuses on monitoring and controlling only one or few chronic diseases without paying attention to a user's overall health conditions such as physical, hearing, or visual strengths. As a result, a user may not be able to properly use the device, and/or the device may miss detecting the user's health issues that could be detected at early stages through performing one or more further tests.

The systems presented herein can test a user's vital signs and strengths, and detect or predict potential health issues that threaten the user. For example, in an embodiment the system is configured to perform the actions of: receiving examination data related to at least one of gripping power, vision health, or hearing strength of a user, analyzing, by a data processor of the system, the received examination data to determine whether the user is at risk of a health problem, and in response to determining that the user is at risk of the health problem, notifying a health care provider about the risk. The system is configured to provide fluid to or receive fluid from the user's body and analyze the fluid to determine one or more contents included in the fluid. For example, the system can be a peritoneal dialysis system or a hemodialysis system. The system can receive blood from the user to dialyze the blood. In some examples, the examination data can be received before allowing the user to use the personal health care system for dialysis.

In some embodiments, the system periodically requests the examination data from the user.

In some embodiments, analyzing the data includes the actions of: comparing the examination data to health data ranges stored in a lookup table that maps a plurality of health data ranges to a plurality of risk categories, each health data range being associated with one or more respective risk categories; and determining that the examination data fits into a particular risk category in the lookup table. Notifying the health care provider about the risk can include providing an indication of the risk category in a message or alert sent to the health care provider. The actions can further include determining that the particular risk category includes a plurality of potential risk factors, and requesting at least a second test on the user to determine which risk factor from among the plurality of potential risk factors threatens the user. Examples of the second test include, but are not limited to a blood test, a urine test, or a physical bodily test. Examples of the one or more risk factors include, but are not limited to a stroke, a heart attack, or an internal or external bleeding.

In some embodiments, analyzing the examination data includes the actions of: comparing the examination data to past health data of the user that is stored on a data storage of the personal health care system to determine a difference between the past health data and the examination data; and determining, based on the difference, one or more risk factors that have a likelihood of threatening the user, the likelihood being greater than a specified threshold value. Notifying the health care provider can include providing an indication of the one or more risk factors in a message or alert sent to the health care provider.

In some systems, the examination data related to gripping power is received through one or more sensors implemented on or within a gripping structure of the personal health care system so that when the user applies force to at least part of the gripping structure, the sensors send the examination data related to gripping power to the data processor. The gripping structure is in the form of at least one of a handle or a squeezable element. In some systems, the examination data related to gripping power is received through a dynamometer attached to the personal health care system, the dynamometer being in communication with the data processor.

In some embodiments, receiving the examination data related to hearing strength includes the actions of: playing a sound to the user, the sound containing more than one frequency; receiving feedback from the user indicating what the user has heard from the played sound; and determining the examination data related to hearing strength from the received feedback. Receiving the feedback can include receiving, from the user, a selection of an item of a plurality of items displayed on a screen of the personal health care system, where one item of the plurality of items provides a correct indication of the played sound.

The played sound can include a speech played to the user. The user may provide the feedback by speaking the speech. The actions can further include performing a speech recognition method on the spoken speech to determine words spoken by the user. The examination data related to hearing strength can be determined by comparing the determined words spoken by the user and the speech included in the played sound.

In some embodiments, receiving the examination data related to hearing strength includes the actions of: playing a sound to the user; receiving a volume level selected by the user, the volume level indicating a volume at which the user comfortably hears the sound; and predicting, based on the received volume level, a volume needed to alert the user when the user is asleep.

In some embodiments, the actions further include presenting one or more symbols on a display of the personal health care system, wherein the examination data related to vision health includes feedback from the user indicating what the user sees on the screen.

Systems and computer-readable medium storing one or more instructions executable by a computer system to perform the above-identified actions and other actions described below are presented herein.

The details of one or more implementations of the present disclosure are set forth in the accompanying drawings and the description below. Other features and advantages of the present disclosure will be apparent from the description and drawings, and from the claims.

### DESCRIPTION OF DRAWINGS

FIG. 1 depicts an example application of an example personal health care system according to the present disclosure.

FIG. 2A depicts a peritoneal dialysis (PD) system as an example medical machine that includes the personal health care system depicted in FIG. 1.

FIG. 2B depicts a hemodialysis (HD) system as an example medical machine that includes the personal health care system depicted in FIG. 1.

FIGS. 3A through 3D depict example test modules of a personal health care system according to implementations of the present disclosure.

FIG. 4 depicts an example process that can be executed according to implementations of the present disclosure.

FIG. 5 is a block diagram of an example computing system according to implementations of the present disclosure.

Like reference symbols in the various drawings indicate like elements.

### DETAILED DESCRIPTION

Implementations of the present disclosure include computer-implemented methods for evaluating health of a user by using a personal health care system (the “system”) that the user may use routinely for her chronic diseases. The personal health care system can be used outside a medical facility or clinic, for example, at the user's house, office, room, etc. The system is capable of evaluating health of the user and determining and/or predicting health risks that the user is currently facing or will likely face in the future (e.g., the near-term future, the long-term future, etc.). The system may be primarily used for other purposes such as treating or measuring data related to one or more particular chronic diseases, and may be used for purposes such as performing the health analysis of the user as a secondary function. Examples of the system can be, but are not limited to, a dialysis machine, a respirator, a scale, a blood pressure testing device, or a wheel chair, to name a few. The personal health care system can be used by one or more users.

The system evaluates the user's health through one or more tests such as a grip test, a hearing test, a speech test, a vision test, etc. The system may require the user to go through the tests before, during, or subsequent to a routine check of the user's chronic disease. For example, the system may require the user to perform these tests before allowing the user to use the system for the patient's chronic disease. The system may require the tests periodically (e.g., once every month), after a particular number of uses (e.g., after ten uses), and/or upon detecting an abnormality in a routine check of the user's disease (e.g., upon detecting an infection in the user's blood).

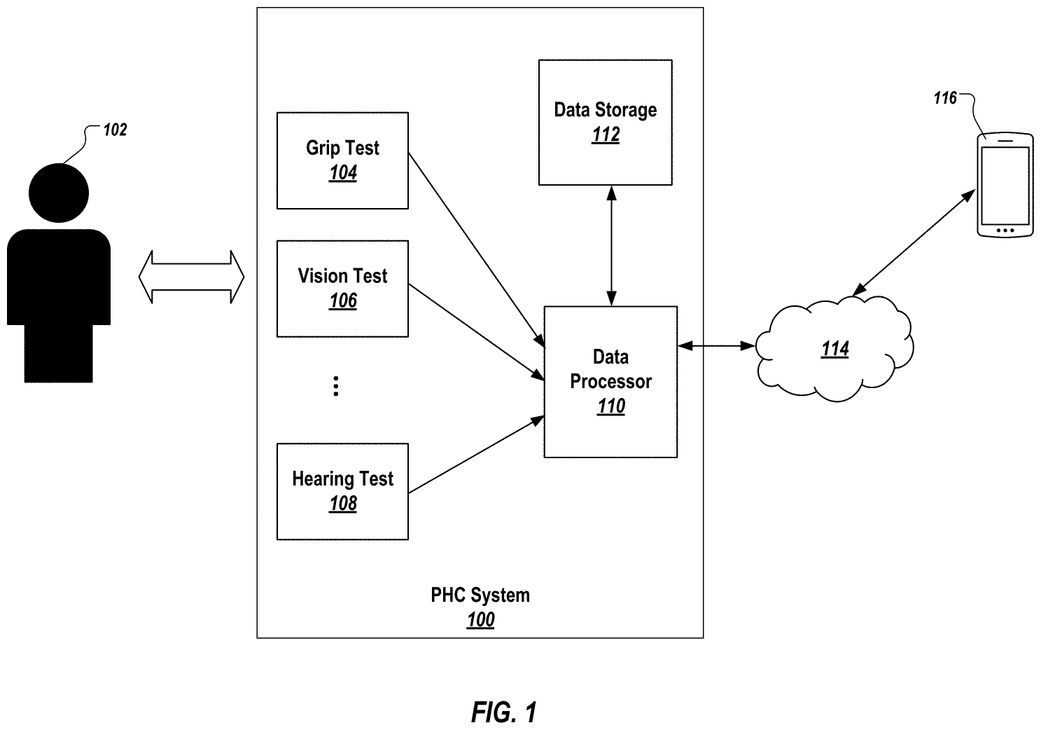

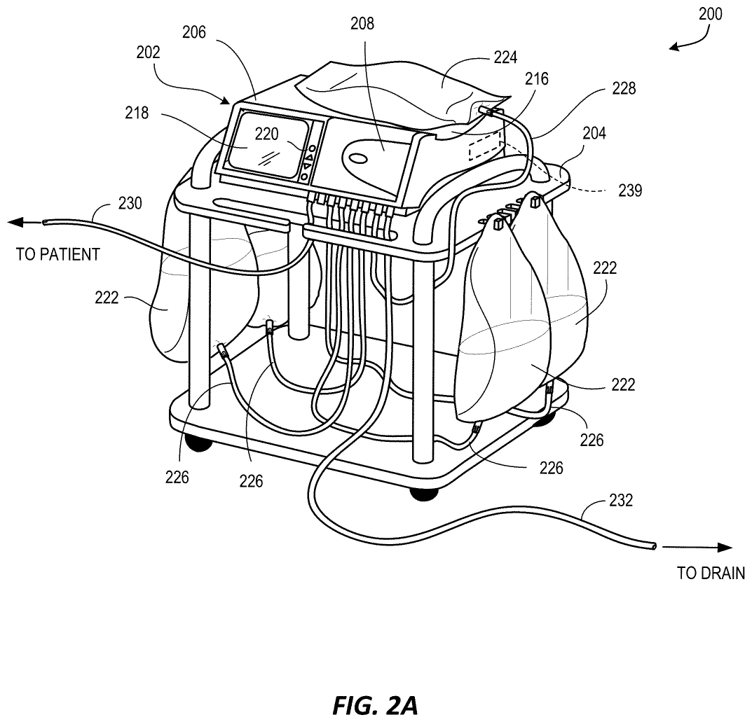

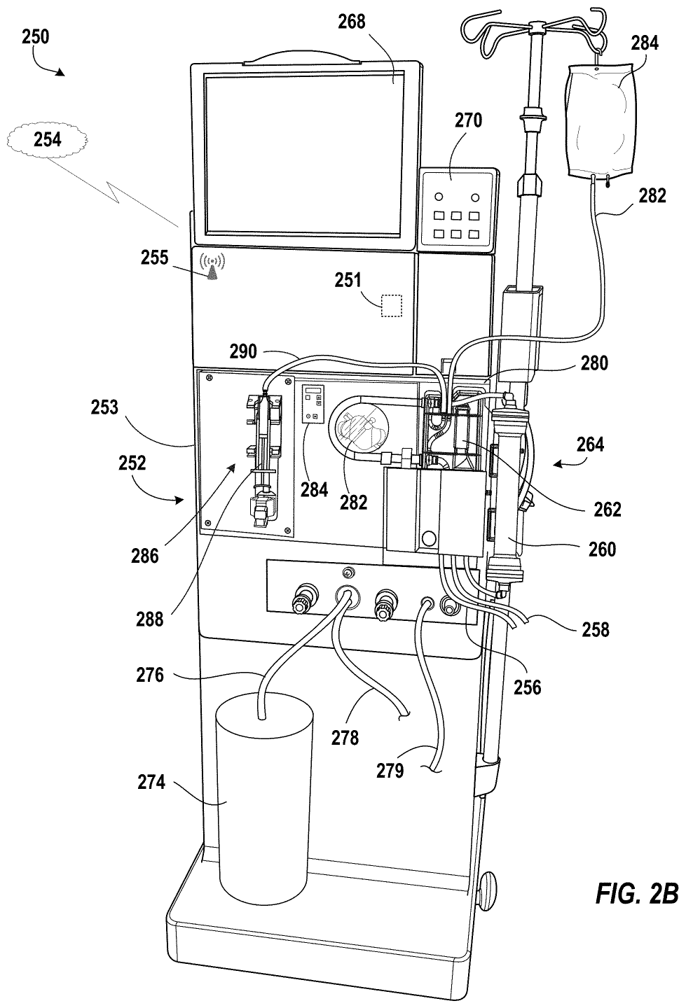

FIG. 1 depicts an example personal health care (PHC) system 100 according to implementations of the present disclosure. FIG. 2A depicts a peritoneal dialysis system 200 and FIG. 2B depicts a hemodialysis system 250 as example medical devices for implementing the PHC system 100. The peritoneal dialysis system 200 and the hemodialysis system 250 are described in detail below.

The PHC system 100 includes one or more test modules that receive, measure, and/or monitor data related to physical capabilities or senses of the user. For example, the PHC system 100 in FIG. 1 includes a grip test module (or dynamometer) 104, a vision test module 106, and a hearing test module 108. One or more additional modules may also be provided.

The test modules receive examination data by testing users' capabilities and senses, and send the examination data to a data processor 110 of the system. The data processor 110 analyzes the examination data to determine whether the user is at risk of a health problem. In cases in which the data processor 110 identifies a health problem or a potential health problem, the data processor 110 notifies the user and/or a health provider about the health risk. For example, the system may present information of the health problem on a display, or may communicate such information to a computing device 116 (e.g., a mobile computing device such as a phone of the user and/or the health care provider) through a network 114.

The PHC system 100 can include a communication unit that can transmit the information to the network 114 or the computing device 116. The communication unit can be a wired or a wireless data transmission unit such as a modem, an electromagnetic antenna, etc.

The health problem can be derived from the examination data directly or indirectly. For example, a directly derived health problem can include at least one of a relatively weak gripping power, a relatively weak vision, or a relatively weak hearing strength, to name a few. In some implementations, one or more of the gripping power, the vision health, or the hearing strength may be compared to a respective threshold value. If the gripping power, vision health, or hearing strength does not satisfy the respective threshold value, the corresponding metric may be determined to be weak (e.g., insufficient). An indirectly derived health problem can include, for example, a heart attack that is detected based on a combination of a weak gripping power and a history of a user's high blood pressure.

### Grip Module

The grip test module (sometimes referred to herein as the “grip module”) 104 performs a grip test on the user. The grip test can be particularly beneficial in detecting a sudden change in the user's power, and/or in predicting the user's capability to properly set up and/or use the PHC system 100 for their chronic disease. For example, upon determining that a gripping power of a user is weaker that a predetermined minimum threshold power, the PHC system 100 may change its default settings according to the user's strength, and/or may alert a health care provider to help the user in setting up the PHC system 100 and/or the medical device that implements the PHC system 100, such as making proper Luer lock tubing connections for blood or dialysate lines.

In some embodiments, the PHC system 100 has child-proof settings that require a user to have a designated amount of strength and/or dexterity to be operable. The system settings may determine how much power would be needed to manipulate (e.g., open, rotate, etc.) the containers and/or switches of the system. Upon determining that a user has a gripping power that does not satisfy the requisite strength and dexterity, the PHC system 100 can change its settings to a less stringent setting that would require less strength for operation than in the child-proof setting.

Upon detecting that the user has shown a sudden weakness in her gripping power, the PHC system 100 may determine that the user is potentially at risk of a health problem. In response, the PHC system 100 may request one or more additional tests from the user, may alert a health care provider about the issue, and/or may contact an emergency health care provider (e.g., call 911).

The grip test module 104 can be implemented within the PHC system 100, or can be attached to the PHC system 100 (e.g., as a dynamometer). The grip test module 104 includes one or more sensors that measure strength of one or more muscles of the user's hand(s). The sensors can be implemented on and/or within a gripping structure of the grip test module 104. When the user holds, pulls, and/or pushes at least part of the gripping structure, the sensors implemented in or within that part of the gripping structure receive data related to the user's hand muscles and transfer the data to the data processor 110.

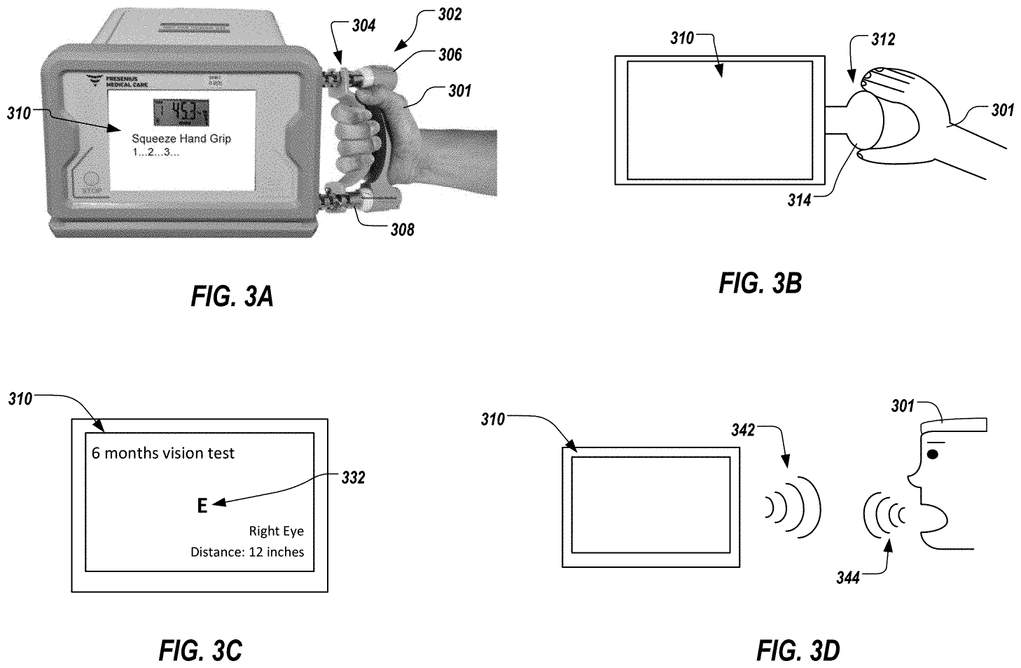

FIGS. 3A and 3B provide example gripping structures 302 and 312. Each of the gripping structures 302, 312 is connected to a screen 310 that can be used to show instructions, such as when and/or how to apply a force on the gripping structures 302, 312, and/or to display a result of the gripping test.

The gripping structure 302 in FIG. 3A has a first handle 304 and a second handle 306. At least one of the first handle 304 and the second handles 306 is movable. For example, when a user 301 pulls the first handle 304 towards the second handle 306, the first handle 304 slides along a shaft 308. One or more sensors can be implemented on any of the handles 304, 306 and/or the shaft 308 to measure the force that the user has applied to move the first handle 304 towards the second handle 306.

The sensors can be located in different parts of the gripping structure 302 to measure strength of different hand muscles. For example, sensors located on the first handle 304 can measure strength of different figures or different parts of respective fingers that hold the first handle 304; sensors on the second handle 306 can measure the strength of the palm or the thumb (e.g., the overall strength of the palm or the thumb or the strengths of particular muscles of the palm or the thumb); and the sensors located on the shaft 308 can measure the overall strength of the user's hand, including the wrist, tendons, etc., that are involved in applying the force on the handles 304, 306.

Alternatively or in addition to the sensors, one or more springs can be implemented within the shaft 308. The user's force can be measured based on the springs' expansion or compression relative to the springs' normal length at their resting position (i.e., when the user does not apply any forces to the handles 304, 306).

The gripping structure 312 of FIG. 3B has a squeezable element 314 and tests the gripping strength of the user 301 based on how much force is applied when the user squeezes the element 314. The element 314 can be made of an elastic material such as rubber that is compressed upon applying a force to it. A difference between a first volume of the element 314 when the element is at rest, and a second volume of the element 314 when the element is under pressure, can correspond to an amount of force that is applied to the element 314. Thus, the difference between the first volume and the second volume can be used to determine the gripping strength of the user. Alternatively, or in addition to being elastic, the gripping structure 312 can include one or more sensors capable of measuring the force applied to the gripping structure 312.

In some embodiments, a combination of the gripping structures 302 and 312 can be used to measure strengths of additional hand muscles and/or tendons. For example, one or both of the handles 304, 306 of the gripping structure 302 can include a squeezable/elastic portion (e.g., similar to the squeezable element 314 of FIG. 3B). Such implementations can be particularly beneficial for measuring the gripping power of people who have weak grips, such as the elderly or people with disabilities. For example, in an embodiment in which the elastic portions of the handles can be squeezed upon applying a relatively weak force as compared to the force needed to move the handles 304, 306 along the shaft 308, the sensors of the elastic portion can detect and measure the force that the user applies to the gripping structure 302 if the user doesn't have enough strength to substantially move either of the handles 304, 306 along the shaft 308.

The gripping structures 302 and 312 transmit their measured data (sometimes referred to herein as the “examination data”) to a processing unit (e.g., the data processor 110 of FIG. 1) capable of measuring the gripping strength of the user. The processing unit can determine an overall gripping strength of the user and/or weaknesses in particular muscles of the user's hand. In some embodiments, in response to determining that one or more particular muscles of the user suffer weaknesses, the processing unit provides particular exercises to strengthen those muscles. In some implementations, a processing unit of the medical machine may be used to measure the gripping strength of the user.

### Vision Module

To utilize the PHC system 100 (for example, at home), a patient often needs to read text such as instructions on a screen of the system, make tubing connections, and/or check fluid samples, for example, to detect abnormalities such as a sign of infection in a blood sample, etc. Some chronic diseases, such as diabetes, cause a variety of eye diseases, which can affect a user's ability to independently utilize the PHC system 100 or a medical machine that implements the PHC system 100 (such as a dialysis machine). Unfortunately, many patients are unaware of their vision impairments and fail to perform a routine check of their visual capabilities.

The vision test module (sometimes referred to herein as the “vision module”) 106 examines the user's vision and sends the examination data to the data processor 110 to test the user's visual capabilities. The data processor 110 evaluates the examination data and alerts the user and/or their health care provider if it detects a substantial impairment or change in the user's visual capabilities. The data processor 110 is capable of determining whether the user requires reading glasses, and suggesting a proper power of the reading glasses to the user. Similar to the other test modules, the vision test module 106 may check the user's vision periodically.

FIG. 3C depicts an example vision test performed by the vision module 106 of an example system according to the present disclosure. The vision test module 106 tests the user's vision by displaying one or more particular characters or symbols 332 on a screen 310 of the system, and requesting a feedback from the user that indicates what the user sees on the screen. The system that implements the vision module 106 may be the PHC system 100 and/or a medical machine that implements the PHC system 100 (e.g., a dialysis system such as the peritoneal dialysis system 200 of FIG. 2A and/or the hemodialysis system 250 of FIG. 2B).

Feedback can be received through the user's spoken words (e.g., “the symbol is facing right”), through a drawing (e.g., of the symbol) that the user makes on a pressure sensitive pad (e.g., a touch screen), or through a user's selection of an answer to a multiple choice question (e.g., press number 1 to indicate that the symbol is facing right.), where only one of the answers provides a correct description of the displayed symbol. Although the symbol 332 is a single character, in some embodiments, the symbol 332 can include multiple characters such as a word or a sentence, and the feedback can include an identification of the multiple characters and/or words. The vision module 106 sends the user's feedback along with associated testing data (e.g., a size of the symbol presented to the user) to the data processor 110 for evaluation.

The vision module 106 may start the vision test by showing a relatively large symbol and reducing the size of the symbol each time that it receives a correct response from the user, until the user is no longer capable of providing the right answer. At this point, the vision module 106 can stop the respective test and send the examination data to the data processor 110 to evaluate and provide a final vision result. The final result of the test can be stored and/or provided to the user or a health care provider.

In cases in which the data processor 110 detects a particular issue in the user's visual capabilities, the data processor 110 notifies the health care provider of the particular issue, and/or performs further tests on the user. For example, detecting cloudiness of an effluent is particularly important in home dialysis contexts because a cloudy effluent indicates potential infection in the user's peritoneum. In cases in which the data processor 110 determines that the user's vision is incapable of properly detecting cloudiness, the dialysis system may initiate a protocol for a more intense scrutiny of the dialysate effluent than its default protocol. Such an intense protocol may include asking questions from the user about how she feels with regard to infection symptoms, asking the user to hold a different colored background behind a tubing window of the dialysis system, requesting the user to ask others to help her review cloudiness of the effluent, and/or notifying the user's health care provider (e.g., a home therapies nurse) about the user's need for assistance in detecting cloudiness in the effluent.

The vision module 106 may perform different tests to measure different visual capabilities of the user, including nearsightedness, light sensitivity, color blindness, farsightedness, etc. Each test can be performed by showing a respective image or video related to the test to the user. For example, an image used for testing the nearsightedness may differ from an image used for testing for color blindness.

The vision module 106 may require the user's head to be at a particular distance from the screen 332 for each of the vision tests described herein. For example, the vision module 106 may require the user to be 12 inches from the screen 310 to test the nearsightedness, 20 inches from the screen 310 to test for light sensitivity, etc.

The PHC system 100 may have a physical distance indicator element such as a measuring stick to help guide the user in determining her distance from the screen 310. The distance indicator can be included in a package of the system as a separate element for the user to use, or can be located on a part of the system such as an edge of a card the system sits upon. Some embodiments can have one or more range-finder technologies such as a camera, a laser, or an ultrasonic position sensor build into the system to detect the user's distance from the screen 310.

### Hearing Module

Hearing capabilities are important for a user to properly receive the PHC system's 100 alerts or spoken instructions. Patients with particular diseases are more likely to suffer from hearing issues. For example, people who suffer from diabetes and kidney failures are twice as likely to suffer from hearing loss. Hearing impairment makes it difficult for a patient to hear and respond to audible alarms or spoken words of a home dialysis machine. Being unaware of her hearing issues, the user may cease using the home dialysis machine.

To resolve such problems, the PHC system 100 includes a hearing test module (sometimes referred to herein as the “hearing module”) 108 that can periodically test the hearing capabilities of the user and adjust its settings accordingly. Working in communication with the data processor 110, the hearing module 108 can test the user's hearing strength by determining the volume and/or the frequencies that the user can hear and the volume and/or frequencies that the user has trouble hearing. The hearing module can perform a variety of hearing tests such as tone testing, speech testing, particular alarm testing, particular warning testing, or particular notification testing, etc.

FIG. 3D depicts an example interaction between the user 301 and the hearing module 108 of an example PHC system 100 according to the present disclosure. As depicted, the hearing module 108 examines the user's hearing capabilities by playing a sound 342 to the user, and receiving a response 344 from the user that indicates what the user heard from the sound 342. The hearing module 108 sends the examination data (including information related to the played sound 342 and the received response 244) to the data processor 110 for evaluation. Upon evaluating the examination data, the data processor 110 determines whether the user has a hearing problem, and if she does, automatically adjusts the system's volume and sound settings, and/or notifies a health care provider of the user's hearing issues.

The sound 342 can be in form of speech and/or a music including a range of frequencies. The response 344 received from the user can be in the form of a voice (e.g., repeating what the user heard from the sound 342), text entered or typed by the user, and/or the user's selection of an answer from multiple answers presented to the user, where only one of the answers indicates a correct answer. For example, the multiple answers can include a buzzing sound, a ringing sound, a rattling sound, particular words, etc.

In general, the hearing module 108 can determine a minimum volume that a user can hear at one or more particular frequencies by playing a sound 342 in those particular frequencies at a first volume and continuously reducing the volume until it reaches a second volume at which the user indicates that she can no longer hear or discern the sound 342. The hearing module 108 (or the data processor 110) can mark the second volume as a minimum playable volume for the user, and the second volume can be used as an indicator to determine the user's hearing strength.

In some embodiments, the hearing module 108 plays the sound 342 in different frequencies to detect one or more frequencies that are difficult for the user to hear. For example, the hearing module 108 may play different sentences or words in different frequencies. Upon detecting that the user has missed identifying a sentence or a word played in a particular frequency, the data processor 110 can determine that the user has trouble hearing that particular frequency. In turn, the data processor 110 can cause the system to avoid using that particular frequency in subsequent alerts, notifications, and/or instructions.

The data processor 110 can detect missed words/sentences in the user's response 344 by performing a speech recognition technique on the response 344. For example, the hearing module 108 can play a speech through the sound 342 and request the user to repeat the speech. The response 344 reflects what the user heard in the sound 342. The hearing module 108 receives the response 344 (e.g., through a microphone) and sends the response 344 to the data processor 110. The data processor 110 performs a speech recognition technique on the data of the response 344 to determine the words spoken by the user. The data processor 110 evaluates hearing accuracy (e.g., hearing strength) of the user by comparing the speech recognized from the data of the response 344 and the actual words included in the speech of the sound 342.

In some embodiments, the hearing module 108 plays a continuous sound, and in response, receives a volume level selected by the user. The volume level indicates a volume that the user comfortably hears the sound. Using the user's selected volume, the data processor 110 can predict a greater volume needed to alert the user when the user is asleep. Such prediction can be performed based on one or more of a difference in volume that would be needed to alert an average person when awake compared to when asleep, the user's particular inputs indicating how heavy of a sleeper the user is, a history of hearing tests and/or alarms performed on the user during different times of the day/night and how the user reacted to them, etc. In cases in which the data processor 110 determines that the system is not capable of producing a volume predicted to be needed for when the user is asleep, the system informs a health care provider to take the responsibility of alerting the user when the user is asleep.

The system may detect that the user is asleep through one or more sensors that monitor the user's metabolism, heart rate, breathing rate, activities, a time of day or night, and/or the user's normal sleeping schedule. The sensors can be attached to the user, for example, as a wristband, a neckless, etc.

In some implementations, one or more other techniques may be used for detecting a required volume for alerting a sleeping user. In some examples, in response to detecting that the user is asleep, the system may start an alarm at a relatively low volume (e.g., compared to a volume that the system uses when the user is awake) and slowly increases the volume until the user reacts to the alarm. The system can mark a volume associated with the point when the user reacts as the volume needed to alert the user when she is sleep.

### Data Processor

As explained above, the data processor 110 evaluates the user's health by analyzing the examination data received from one or more test modules. The data processor 110 can do so by comparing the examination data to reference data stored on a data storage 112. While the data processor 110 is shown and described as belonging to the PHC system 100, it should be understood that a data processor of a medical machine (e.g., a dialysis system) may also or instead be used to analyze the examination data received from the one or more test modules.

The reference data can be provided to the data storage 112 as default health information, for example, at a manufacturing stage of the PHC system 100, or through a communication between the PHC system 100 and a source database through a network (e.g., the Internet). For example, the reference data may map biological information (e.g., sex, weight, height, age, history of particular chronic diseases, etc.) of an average person that is biologically similar to the user, to determine expected grip, hearing, or vision strength of the user. The reference data may include prior examination data received from testing the user's health by the aforementioned test modules in the past. The storage data may maintain this information separately, or may use this information to tune the default health information stored in the data storage 112.

The data storage 112 can store a lookup table mapping a plurality of health data ranges to a plurality of risk categories. The health data ranges may be defined in the reference data. Each health data range can be associated with one or more respective risk categories. Upon receiving the examination data, the data processor 110 can compare the received examination data with the data stored in the lookup data to determine whether a particular health risk threatens the user.

For example, the data storage 112 may store health information related to a male user aged between 55-60 years of age with diabetes as reference data associated with a first user of the system. The lookup table may list a vision strength between 20/20 and 20/100 as a first health data range, a vision strength between 20/100 and 20/200 as a second health data range, and a vision strength over 20/200 as a third health data range. The lookup table may map the first health data range to a normal vision strength, the second health data range to diminished vision strength, and the third health data range to a dangerously low vision strength. In cases in which the received examination data indicates that the vision strength is diminished or dangerously low, the data processor 110 can alert the user or their health care provider about a potential health risk.

In some examples, the lookup table lists which particular health risks are potentially threatening the user. The health risks can be the risks that are derived directly from the data processor's 110 evaluation of the examination data. For example, the health risks can include a visual impairment or can include risks that are derived indirectly such as an unsatisfactory level of blood sugar or blood pressure.

In some examples, the data processor 110 may determine that a grip strength below 80 pounds is insufficient for a male user aged between 40-45 years, and may be indicative of a potential health risk. Based on a comparison of the examination data received from the test modules and the data stored in the lookup table, the data processor 110 can identify health risk categories (e.g., lack of sleep or exercise, sickness, emergency situations, blood circulation issues, etc.) that potentially threaten the user.

In some embodiments, the data processor 110 may combine health data ranges listed in the lookup table with health history of the user to determine potential health risks more precisely. For example, the data storage 112 may include information indicative of a history of high blood pressure for the male user continuing with the example above. Upon receiving an indication of a relatively low grip strength, the data processor 110 may determine that something is wrong with blood circulation in the user's body. The data processor 110 can send a message to the user or the health care provider notifying them of the potential blood circulation issue. The message can be in the form of a text message, an alert, a phone call, etc.

Based on prior tests, the data processor 110 may determine that in general, the user has a weak grip, a vision that requires reading glasses of a particular power, or a hearing ability that requires a volume of a particular decibel. In determining health risks that threaten the user, the data processor 110 compares the currently (i.e., most recently) received examination data to the health history of the user to determine what is normal for this particular user. In cases in which the data processor 110 determines an abnormality (e.g., an unusually weak grip, hearing power, or vision), the data processor 110 determines potential health risk factors that may threaten the user.

For example, a test history may indicate that a user's strength has gradually weakened over a period of time. Based on how fast the weakness has happened and/or over what period of time the decrease in the weakness has happened, the data processor 110 may determine that the weakness is a result of the user's age, or a consequence of a particular health risk, and notify the user or the health care provider accordingly.

Each of the potential health risks (or health risk categories) that the data processor 110 identifies may include multiple risk factors. For example, continuing with the example above, the health risk category associated with blood circulation may include risk factors of: stroke, heart attack, internal/external bleeding, high/low blood pressure, high/low blood sugar, etc. The data processor 110 can request further tests from the user to identify one or more particular risk factors that are more likely than the other risk factors to threaten the user. For example, the data processor 110 may request a blood test, a urine test, or a physical bodily test, or ask some questions from the user to identify risk factors that are likely. Based on the results of such tests, the data processor 110 can identify one or more likely risk factors and send indications of the one or more likely risk factors to the user or a health care provider.

For each health risk factor, the data processor 110 may determine a respective likelihood of the risk factor threatening the user. The data processor 110 can select the risk factors that have the highest threatening likelihood and send indications of those selected risk factors to the user or a health care provider. For example, the data processor 110 may select a risk factor that has a likelihood higher than a particular threshold value as dangerous and more threatening that risk factors that have respective likelihoods less than the threshold value.

In some implementations, each health risk category (or risk factor) is associated with a respective threshold value and is compared to its respective threshold value to determine whether it is a dangerous (or a threatening) health risk factor for the user. For example, a stroke risk may have a threshold value of 40% and a bleeding risk may have a threshold value of 70%. Accordingly, when the system determines that there is a 50% stroke risk for a user (or 50% likelihood that the user is having or will soon have a stroke), the system determines that this risk is greater than the threshold value specified for stroke and notifies the user or a health care provider that a stroke is likely threatening the user. In the same example, when the system determines that there is a 60% bleeding risk (i.e., likelihood of bleeding) for the user, the system determines that this likelihood is less than the specified threshold value for bleeding risk, and will not determine bleeding as a risk that is threatening the user.

In concluding that a particular health risk threatens the user, the data processor 110 may compare the received examination data with the user's health history to determine a trend or a continuation in receiving failed tests or relatively weak test results. For example, an impatient user may try to skip the tests, for example, by providing random responses such as applying a random pressure on the gripping module 312, and/or providing random answers to the hearing module 108 or vision module 106 instead of following the modules' instructions. Upon detecting a trend in receiving the random responses, the data processor 110 may identify a health care provider to discuss the issue with the use or to help the user figure out how to use the system and its tests.

### Example Medical Systems

Although the present techniques are described with reference to dialysis machines including peritoneal dialysis machines and hemodialysis machines, the disclosed health evaluation can be performed by any medical devices used for treatment, monitoring, or assisting a user with one or more chronic diseases.

The peritoneal dialysis (PD) system 200 in FIG. 2A and the hemodialysis (HD) system 250 in FIG. 2B show example medical devices for implementing the personal health care system 100 of FIG. 1. For example, the PHC system 100 can be part of the PD machine 202 in the PD system 200, can be part of the HD machine 252 of the HD system 250, or can be in communication with the PD machine 202 or the HD machine 252, for example, through a transceiver 255.

In some implementations, the PD system 200 is configured for use at a patient's home (e.g., a home PD system). In some implementations, the HD system 250 is configured for use at a patient's home (e.g., a home HD system).

The peritoneal dialysis system 200 includes a PD machine (also generally referred to as a PD cycler) 202 seated on a cart 204. The PD machine 202 includes a housing 206, a door 208, and a cassette interface 210 that contacts a disposable PD cassette 212 when the cassette 212 is disposed within a cassette compartment 214 formed between the cassette interface 210 and the closed door 208. A heater tray 216 is positioned on top of the housing 206. The heater tray 216 is sized and shaped to accommodate a bag of PD solution such as dialysate (e.g., a 5 liter bag of dialysate). The PD machine 202 also includes a user interface such as a touch screen display 218 and additional control buttons 220 that can be operated by a user (e.g., a caregiver or a patient) to allow, for example, set up, initiation, and/or termination of a PD treatment.

Dialysate bags 222 are suspended from fingers on the sides of the cart 204, and a heater bag 224 is positioned in the heater tray 216. The dialysate bags 222 and the heater bag 224 are connected to the cassette 212 via dialysate bag lines 226 and a heater bag line 228, respectively. The dialysate bag lines 226 can be used to pass dialysate from dialysate bags 222 to the cassette 212 during use, and the heater bag line 228 can be used to pass dialysate back and forth between the cassette 212 and the heater bag 224 during use. In addition, a patient line 230 and a drain line 232 are connected to the cassette 212. The patient line 230 can be connected to a patient's abdomen via a catheter and can be used to pass dialysate back and forth between the cassette 212 and the patient's peritoneal cavity during use. The catheter may be connected to the patient line 230 via a port such as a fitting. The drain line 232 can be connected to a drain or drain receptacle and can be used to pass dialysate from the cassette 212 to the drain or drain receptacle during use.

The PD machine 202 also includes a control unit 239 (e.g., a processor). The control unit 239 can receive signals from and transmit signals to the touch screen display 218, the control panel 220, and the various other components of the PD system 200. The control unit 239 can control the operating parameters of the PD machine 102. In some implementations, the control unit 239 is an MPC823 PowerPC device manufactured by Motorola, Inc.

The HD system 250 illustrated in FIG. 2B includes an HD machine 252 to which a disposable blood component set 264 that forms a blood circuit is connected. During hemodialysis, arterial and venous patient lines 256, 258 of the blood component set 264 are connected to a patient and blood is circulated through various blood lines and components, including a dialyzer 260, of the blood component set 264. At the same time, dialysate is circulated through a dialysate circuit formed by the dialyzer 260 and various other dialysate components and dialysate lines connected to the HD machine 252. Many of these dialysate components and dialysate lines are located inside the housing 253 of the HD machine 252, and are thus not visible in FIG. 2B. The dialysate passes through the dialyzer 260 along with the blood. The blood and dialysate passing through the dialyzer 260 are separated from one another by a semi-permeable structure (e.g., a semi-permeable membrane and/or semi-permeable microtubes) of the dialyzer 260. As a result of this arrangement, toxins are removed from the patient's blood and collected in the dialysate. The filtered blood exiting the dialyzer 260 is returned to the patient. The dialysate that exits the dialyzer 260 includes toxins removed from the blood and is commonly referred to as “spent dialysate.” The spent dialysate is routed from the dialyzer 260 to a drain.

One of the components of the blood component set 264 is an air release device 262. The air release device 262 includes a self-sealing vent assembly that allows air to pass through while inhibiting (e.g., preventing) liquid from passing through. As a result, if blood passing through the blood circuit during treatment contains air, the air will be vented to atmosphere as the blood passes through the air release device 262.

As shown in FIG. 2B, a dialysate container 274 is connected to the HD machine 252 via a dialysate supply line 276. A drain line 278 and an ultrafiltration line 279 also extend from the HD machine 252. The dialysate supply line 276, the drain line 278, and the ultrafiltration line 279 are fluidly connected to the various dialysate components and dialysate lines inside the housing 253 of the HD machine 252 that form part of the dialysate circuit. During hemodialysis, the dialysate supply line 276 carries fresh dialysate from the dialysate container 274 to the portion of the dialysate circuit located inside the HD machine 252. As noted above, the fresh dialysate is circulated through various dialysate lines and dialysate components, including the dialyzer 260, that form the dialysate circuit. As the dialysate passes through the dialyzer 260, it collects toxins from the patient's blood. The resulting spent dialysate is carried from the dialysate circuit to a drain via the drain line 278. When ultrafiltration is performed during treatment, a combination of the spent dialysate and excess fluid drawn from the patient is carried to the drain via the ultrafiltration line 279.

The blood component set 264 is secured to a module 280 attached to the front of the HD machine 252. The module 280 includes a blood pump 282 capable of driving blood through the blood circuit. The module 280 also includes various other instruments capable of monitoring the blood flowing through the blood circuit. The module 280 includes a door that when closed, as shown in FIG. 2B, cooperates with the front face of the module 280 to form a compartment sized and shaped to receive the blood component set 264. In the closed position, the door presses certain blood components of the blood component set 264 against corresponding instruments exposed on the front face of the module 280. Such an arrangement facilitates control of the flow of blood through the blood circuit and monitoring of the blood flowing through the blood circuit.

The blood pump 282 can be controlled by a blood pump module 284. The blood pump module 284 includes a display window, a start/stop key, an up key, a down key, a level adjust key, and an arterial pressure port. The display window displays the blood flow rate setting during blood pump operation. The start/stop key starts and stops the blood pump 282. The up and down keys increase and decrease the speed of the blood pump 282. The level adjust key raises a level of fluid in an arterial drip chamber.

A drug pump 286 also extends from the front of the HD machine 252. The drug pump 286 is a syringe pump that includes a clamping mechanism configured to retain a syringe 288 of the blood component set 264. The drug pump 286 also includes a stepper motor configured to move the plunger of the syringe 288 along the axis of the syringe 288. A shaft of the stepper motor is secured to the plunger in a manner such that when the stepper motor is operated in a first direction, the shaft forces the plunger into the syringe 288, and when operated in a second direction, the shaft pulls the plunger out of the syringe 288. The drug pump 286 can thus be used to inject a liquid drug (e.g., heparin) from the syringe 288 into the blood circuit via a drug delivery line 290 during use, or to draw liquid from the blood circuit into the syringe 288 via the drug delivery line 290 during use.

The HD machine 252 includes a touch screen 268 and a control panel 270. The touch screen 268 and the control panel 270 allow an operator to input various treatment parameters to the HD machine 252 and to otherwise control the HD machine 252. In addition, the touch screen 268 serves as a display. The touch screen 268 functions to provide information to the patient and the operator of the HD system 250. For example, the touch screen 268 may display information related to a dialysis treatment to be applied to the patient, including information related to a prescription, as described above.

The HD machine 252 includes a processing module 251 that resides inside the machine and which is configured to communicate with the touch screen 268 and the control panel 270. The processing module 251 is configured to receive data from the touch screen 268 and the control panel 270 and control the HD machine 252 based on the received data. For example, the processing module 251 can adjust the operating parameters of the HD machine 252.

The HD machine 252 is configured to connect to a network 254. The HD machine 252 includes a transceiver 255 that is configured to facilitate the connection to the network 254. Other medical devices (e.g., peripheral devices or monitors, other dialysis machines, etc.) may be configured to connect to the network 254 and communicate with the HD machine 252. Similarly, one or more remote entities, such as issuers of digital prescription files and/or authority services tasked with verifying identities of issuers and certifying ownership of public keys corresponding to the issuers, may be able to connect to the network 254 and communicate with the HD machine 252 in order to provide digital prescriptions for implementing on the HD machine 252, digital certificates, and/or public keys usable to check digital signatures, as described above.

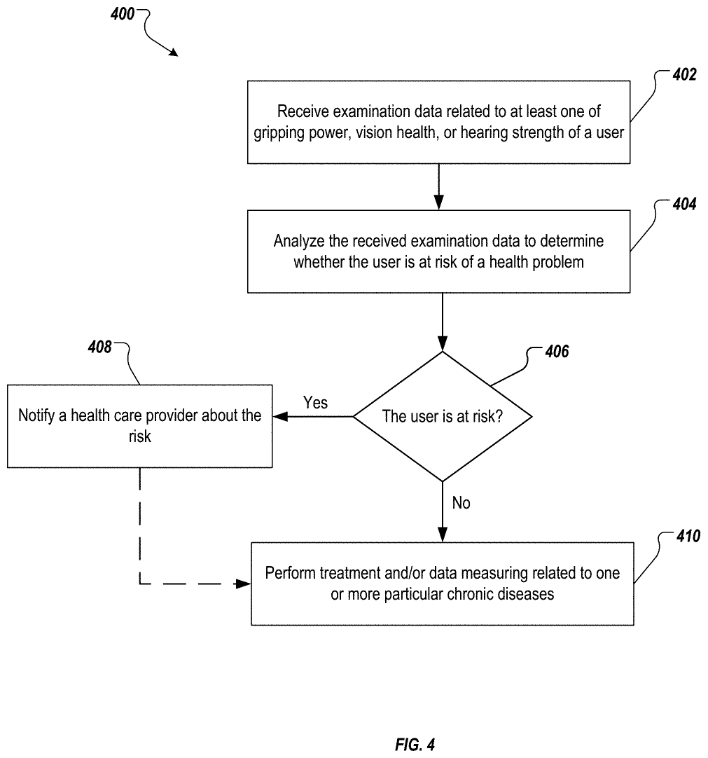

FIG. 4 depicts an example process 400 that can be executed according to implementations of the present disclosure. The process 400 can be performed by any of the PHC systems described herein, including the PHC system 100 of FIG. 1 and/or a medical machine/system that implements the PHC system 100.

In process 400, examination data is received from a user (402). The examination data includes at least one of a gripping power, a vision health, or a hearing strength of the user, and is received through a test module of a personal health care system. Examples of such modules and systems are provided in FIG. 1.

The examination data is analyzed to determine whether the user is at risk of a health problem (404). For example, a data processor can receive the examination data from respective test modules and analyze the data. The data can be analyzed by comparing it to reference data and/or a health history of the user.

In response to determining that the user is at risk of a health issue (406), a health care provider is notified about the risk (408). For example, the data processor may order a communication unit of the personal health care system to send a message or an alert to the health care provider. The message or the alert can include information of the specificities of the identified health risk. Alternatively or in addition, the user herself may be informed of the risk, for example, by displaying an alert or a message on a screen of the system.

Subsequent to the testing processes, a treatment and/or data measuring related to one or more particular chronic diseases can be performed on the user (410). For example, in an example of the personal health care system being a dialysis machine, the machine advances to dialyzing the user's blood upon finishing the test processes. In some embodiments, the dialysis procedure can be performed before or concurrently with performing the tests.

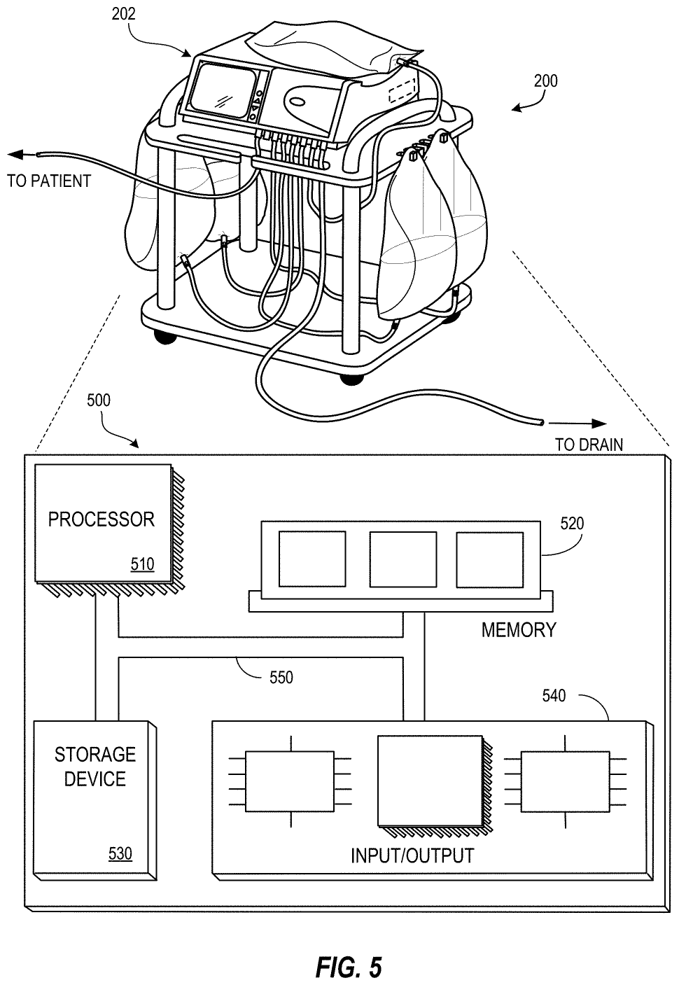

FIG. 5 is a block diagram of an example computer system 500 that can be used as part of the PHC system 100, for example, in connection with the peritoneal dialysis system 200 and/or the hemodialysis system 250. A control unit, a computing device, and/or a microcontroller could be examples of the system 500 described here. The system 500 includes a processor 510, a memory 520, a storage device 530, and an input/output device 540. Each of the components 510, 520, 530, and 540 can be interconnected, for example, using a system bus 550. The processor 510 is capable of processing instructions for execution within the system 500. The processor 510 can be a single-threaded processor, a multi-threaded processor, or a quantum computer. The processor 510 is capable of processing instructions stored in the memory 520 or on the storage device 530. The processor 510 may execute operations such as causing the dialysis system to carry out dialysis functions.

The memory 520 stores information within the system 500. In some implementations, the memory 520 is a computer-readable medium. The memory 520 can, for example, be a volatile memory unit or a non-volatile memory unit. In some implementations, the memory 520 stores information for causing the pumps of the dialysis system to operate as described herein.

The storage device 530 is capable of providing mass storage for the system 500. In some implementations, the storage device 530 is a non-transitory computer-readable medium. The storage device 530 can include, for example, a hard disk device, an optical disk device, a solid-date drive, a flash drive, magnetic tape, or some other large capacity storage device. The storage device 530 may alternatively be a cloud storage device, e.g., a logical storage device including multiple physical storage devices distributed on a network and accessed using a network.

The input/output device 540 provides input/output operations for the system 500. In some implementations, the input/output device 540 includes one or more of network interface devices (e.g., an Ethernet card), a serial communication device (e.g., an RS-232 10 port), and/or a wireless interface device (e.g., an 802.11 card, a 3G wireless modem, or a 4G wireless modem). In some implementations, the input/output device 540 may include short-range wireless transmission and receiving components, such as Wi-Fi, Bluetooth, and/or near field communication (NFC) components, among others. In some implementations, the input/output device includes driver devices configured to receive input data and send output data to other input/output devices, e.g., keyboard, printer and display devices (such as the touch screen display 118). In some implementations, mobile computing devices, mobile communication devices, and other devices are used.

In some implementations, the system 500 is a microcontroller. A microcontroller is a device that contains multiple elements of a computer system in a single electronics package. For example, the single electronics package could contain the processor 510, the memory 520, the storage device 530, and input/output devices 540.

A number of implementations of the present disclosure have been described. Nevertheless, it will be understood that various modifications may be made without departing from the spirit and scope of the present disclosure. Accordingly, other implementations are within the scope of the following claims.

What is claimed is:

1. A computer-implemented method of evaluating health of a user, the method comprising:
receiving, by a personal health care system, examination data related to at least one of gripping power, vision health, or hearing strength of a user, wherein the personal health care system is configured to provide fluid to or receive fluid from the user's body and analyze the fluid to determine one or more contents included in the fluid;
analyzing, by a data processor of the personal health care system, the received examination data to determine whether the user is at risk of a health problem; and
in response to determining that the user is at risk of the health problem, notifying a health care provider about the risk.

2. The method of claim 1, wherein the personal health care system is a peritoneal dialysis system or a hemodialysis system.

3. The method of claim 2, wherein the personal health care system receives blood from the user to dialyze the blood, and wherein the examination data is received before allowing the user to use the personal health care system for dialysis.

4. The method of claim 1, further comprising periodically requesting the examination data from the user.

5. The method of claim 1, wherein analyzing the data comprises:
comparing the examination data to health data ranges stored in a lookup table, the lookup table mapping a plurality of health data ranges to a plurality of risk categories, each health data range being associated with one or more respective risk categories; and
determining that the examination data fits into a particular risk category in the lookup table,
wherein notifying the health care provider about the risk comprises providing an indication of the risk category in a message or alert sent to the health care provider.

6. The method of claim 5, further comprising:
determining that the particular risk category includes a plurality of potential risk factors; and
requesting at least a second test on the user, the second test for determining which risk factor from among the plurality of potential risk factors threatens the user.

7. The method of claim 6, wherein the second test includes a blood test, a urine test, or a physical bodily test.

8. The method of claim 6, wherein the one or more risk factors include at least one of a stroke, a heart attack, or an internal or external bleeding.

9. The method of claim 1, wherein analyzing the examination data comprises:
comparing the examination data to past health data of the user that is stored on a data storage of the personal health care system to determine a difference between the past health data and the examination data; and
determining, based on the difference, one or more risk factors that have a likelihood of threatening the user, the likelihood being greater than a specified threshold value,
wherein notifying the health care provider comprises providing an indication of the one or more risk factors in a message or alert sent to the health care provider.

10. The method of claim 1, wherein the examination data related to gripping power is received through one or more sensors implemented on or within a gripping structure of the personal health care system so that when the user applies force to at least part of the gripping structure, the sensors send the examination data related to gripping power to the data processor.

11. The method of claim 10, wherein the gripping structure is in the form of at least one of a handle or a squeezable element.

12. The method of claim 1, wherein the examination data related to gripping power is received through a dynamometer attached to the personal health care system, the dynamometer being in communication with the data processor.

13. The method of claim 1, wherein receiving the examination data related to hearing strength comprises:
playing a sound to the user, the sound containing more than one frequency;
receiving feedback from the user indicating what the user has heard from the played sound; and
determining the examination data related to hearing strength from the received feedback.

14. The method of claim 13, wherein receiving the feedback comprises receiving, from the user, a selection of an item of a plurality of items displayed on a screen of the personal health care system, wherein one item of the plurality of items provides a correct indication of the played sound.

15. The method of claim 13, wherein the played sound comprises a speech played to the user, wherein the user provides the feedback by speaking the speech, and the method further comprises performing a speech recognition method on the spoken speech to determine words spoken by the user, wherein the examination data related to hearing strength is determined by comparing the determined words spoken by the user and the speech included in the played sound.

16. The method of claim 1, wherein receiving the examination data related to hearing strength comprises:
playing a sound to the user;
receiving a volume level selected by the user, the volume level indicating a volume at which the user comfortably hears the sound; and
predicting, based on the received volume level, a volume needed to alert the user when the user is asleep.

17. The method of claim 1, further comprising:
presenting one or more symbols on a display of the personal health care system,
wherein the examination data related to vision health includes feedback from the user indicating what the user sees on the screen.

18. A non-transitory, computer-readable medium storing one or more instructions executable by a computer system of a personal health care system to perform operations comprising:
receiving examination data related to at least one of gripping power, vision health, or hearing strength of a user, wherein the personal health care system is configured to provide fluid to or receive fluid from the user's body and analyze the fluid to determine one or more contents included in the fluid;
analyzing, by a data processor of the personal health care system, the received examination data to determine whether the user is at risk of a health problem; and
in response to determining that the user is at risk of the health problem, notifying a health care provider about the risk.

19. A personal health care system comprising:
one or more testing modules configured to receive examination data related to at least one of gripping power, vision health, or hearing strength of a user;
one or more pumps configured to provide fluid to or receive fluid from the user's body and analyze the fluid to determine one or more contents included in the fluid;
a data processor unit configured to analyze the received examination data to determine whether the user is at risk of a health problem, and
a communication unit capable of communicating information to a computing system,
wherein in response to determining that the user is at risk of the health problem, the data processor requests the communication unit to notify a health care provider about the risk.

20. The system of claim 19, wherein the personal health care system is a peritoneal dialysis system or a hemodialysis system.
